(19) United States
(12) Patent Application Publication
(10) Pub. No.: US 2023/0165718 A1
(43) Pub. Date: Jun. 1, 2023

(54) METHODS, DEVICES, AND SYSTEMS FOR TREATING LENS PROTEIN AGGREGATION DISEASES

(71) Applicant: Alan Neil Glazier, Rockville, MD (US)

(72) Inventor: Alan Neil Glazier, Rockville, MD (US)

(21) Appl. No.: 17/537,088

(22) Filed: Nov. 29, 2021

(51) Int. Cl. A61F 9/008

(52) U.S. Cl. CPC A61F 9/0084

(57) ABSTRACT

Disclosed herein are methods, devices, and systems for treating lens protein aggregation diseases. A system is disclosed that includes a source of light energy that emits one or more beams of light energy, a focuser for focusing the one or more beams into a predetermined area of the lens epithelium, and an adjuster for adjusting at least one parameter of the one or beams. A method is also disclosed that includes focusing one or more beams of light energy from a source of light energy on to a predetermined area of an eye lens, pulsing the one or more beams, scanning the one or more beams, measuring one or more types of radiation from the predetermined area, and utilizing the one or more measured types of radiation to decide whether to stop or adjust the one or more beams.

### FIELD OF THE INVENTION

The application relates generally to methods, devices, and systems for treating lens protein aggregation diseases. In particular, the application relates to novel methods, devices, and systems for laser-mediated treatment of presbyopia and cataracts by managing internal lens pressure.

### BACKGROUND

Mammalian lens protein aggregation diseases affect the human eye, including presbyopia and cataract. For the average healthy (e.g., non-diabetic, non-smoking) individual, presbyopia can manifest clinically in the early 40's as difficulty seeing objects at close range. However, the processes that lead to presbyopia often begins decades before any clinical symptoms are evident. One of the aforementioned processes leads to a dramatic increase in lens stiffness as an individual ages. For instance, the nucleus, which is a part of the eye lens, becomes approximately 500- to 1000-fold stiffer over the average person's lifetime. Generally, the onset of symptoms associated with presbyopia and other lens aggregation diseases are attributed to the loss of natural enzymatic and antioxidant protection in the eye against, for instance, ultraviolet A (UVA) and ultraviolet B (UV B) radiation, with a concurrent increase in the production of photochemically active chromophores (oxidants).

Accordingly, the key cause of presbyopia and, ultimately, cataractogenesis, is believed to be multifactorial, influenced by a combination of endogenous and exogenous oxidation. Endogenous oxidation occurs via internal mechanisms (e.g., intraocular photochemical generation of free radicals and other oxidants), while exogenous oxidation may be due to exposure to environmental causes (e.g., an increased exposure over an individual's lifespan to short wavelength and ultraviolet (UV) radiation, chemical ingestion (such as smoking), diabetes, and the like).

However, the theory for oxidation as the root cause of presbyopia and other mammalian lens aggregation diseases cannot alone account for changes that result in protein aggregation (e.g., an increase in lens pressure, a decrease in lens flexibility). Such changes may play a more significant role in lens aggregation diseases than currently acknowledged.

Given the foregoing, there exists a significant need for novel technology that manages (e.g., maintains and/or reduces) internal lens pressure, thus reducing onset and/or treating lens protein aggregation diseases, such as, for instance, presbyopia and cataracts.

### SUMMARY

It is to be understood that both the following summary and the detailed description are exemplary and explanatory and are intended to provide further explanation of the invention as claimed. Neither the summary nor the description that follows is intended to define or limit the scope of the invention to the particular features mentioned in the summary or in the description.

In general, the present disclosure is directed towards methods, devices, and systems for non-invasively, or minimally invasively, maintaining or reducing internal lens pressure. In particular, the disclosure relates to methods, devices, and systems for using light energy (e.g., emitted from one or more lasers) to disrupt one or more predetermined areas of the lens epithelium, thereby disrupting the lens epithelium's ability to continually produce proteins, thereby lessening compaction and/or internal lens pressure. The one or more predetermined areas may be located in a periphery of the lens epithelium, and, more specifically, in a germinative zone.

In at least one example, a system for non-invasive or minimally invasive photomanipulation of the lens epithelium of an animal or human eye is disclosed. The system includes a source of light energy (e.g., a laser) emitting one or more beams of light energy, a focuser for focusing the one or more beams of light energy into a predetermined area of the lens epithelium, and an adjuster for adjusting at least one parameter (e.g., focus, intensity, wavelength, pulse length, repetition frequency, and/or pulse train length) of the one or beams of light energy.

In at least a further example, a method for non-invasive or minimally invasive photomanipulation of the lens epithelium of an animal or human eye is disclosed. The method includes focusing one or more beams of light energy from a source of light energy (e.g., a laser) on to a predetermined area of an eye lens, pulsing the one or more beams of light energy, scanning the one or more beams of light energy relative to the eye lens, measuring one or more types of radiation from the predetermined area, and utilizing the one or more measured types of radiation to decide whether to stop the one or more beams of light energy, to adjust one or more parameters associated with the one or more beams of light energy, and/or to adjust one or more parameters associated with the scanning.

The one or more parameters associated with the one or more beams of light energy may include, for instance, focus, intensity, wavelength, pulse length, repetition frequency, and/or pulse train length. The one or more parameters associated with the scanning may include, for example, scan velocity, size of scanned volume, scan repetitions, and scan pattern.

The aforementioned method may further comprise adjusting the one or more beams of light energy to obtain cleavage of one or more molecules. These one or more molecules may be, for instance, lens proteins, lens protein cross-links, macromolecular adducts, and the like.

These and further and other objects and features of the invention are apparent in the disclosure, which includes the above and ongoing written specification, as well as the drawings.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIGS. 1A-1B show a system for directing one or more beams of energy from an energy source into the eye (FIG. 1A), including, specifically, directing one or more beams of energy into a portion of the lens epithelium, such as the germinal zones (FIG. 1B), according to at least one example of the present disclosure.

FIG. 2 is a flow diagram of a method for manipulation of lens epithelium, according to at least one example of the present disclosure.

### DETAILED DESCRIPTION

The present invention is more fully described below with reference to the accompanying figures.

The following description is exemplary in that several embodiments are described (e.g., by use of the terms “preferably,” “for example,” or “in one embodiment”); however, such should not be viewed as limiting or as setting forth the only embodiments of the present invention, as the invention encompasses other embodiments not specifically recited in this description, including alternatives, modifications, and equivalents within the spirit and scope of the invention. Further, the use of the terms “invention,” “present invention,” “embodiment,” and similar terms throughout the description are used broadly and not intended to mean that the invention requires, or is limited to, any particular aspect being described or that such description is the only manner in which the invention may be made or used. Additionally, the invention may be described in the context of specific applications; however, the invention may be used in a variety of applications not specifically described.

The embodiment(s) described, and references in the specification to “one embodiment”, “an embodiment”, “an example embodiment”, etc., indicate that the embodiment(s) described may include a particular feature, structure, or characteristic. Such phrases are not necessarily referring to the same embodiment. When a particular feature, structure, or characteristic is described in connection with an embodiment, persons skilled in the art may effect such feature, structure, or characteristic in connection with other embodiments whether or not explicitly described.

In the several figures, like reference numerals may be used for like elements having like functions even in different drawings. The embodiments described, and their detailed construction and elements, are merely provided to assist in a comprehensive understanding of the invention. Thus, it is apparent that the present invention can be carried out in a variety of ways, and does not require any of the specific features described herein. Also, well-known functions or constructions are not described in detail since they would obscure the invention with unnecessary detail. Any signal arrows in the drawings/figures should be considered only as exemplary, and not limiting, unless otherwise specifically noted. Further, the description is not to be taken in a limiting sense, but is made merely for the purpose of illustrating the general principles of the invention, since the scope of the invention is best defined by the appended claims.

It will be understood that, although the terms first, second, etc. may be used herein to describe various elements, these elements should not be limited by these terms. These terms are only used to distinguish one element from another. Purely as a non-limiting example, a first element could be termed a second element, and, similarly, a second element could be termed a first element, without departing from the scope of example embodiments. As used herein, the term “and/or” includes any and all combinations of one or more of the associated listed items. As used herein, “at least one of A, B, and C” indicates A or B or C or any combination thereof. As used herein, the singular forms “a”, “an,” and “the” are intended to include the plural forms as well, unless the context clearly indicates otherwise. It should also be noted that, in some alternative implementations, the functions and/or acts noted may occur out of the order as represented in at least one of the several figures. Purely as a non-limiting example, two figures shown in succession may in fact be executed substantially concurrently or may sometimes be executed in the reverse order, depending upon the functionality and/or acts described or depicted.

As used herein, ranges are used herein in shorthand, so as to avoid having to list and describe each and every value within the range. Any appropriate value within the range can be selected, where appropriate, as the upper value, lower value, or the terminus of the range.

Unless indicated to the contrary, numerical parameters set forth herein are approximations that can vary depending upon the desired properties sought to be obtained. At the very least, and not as an attempt to limit the application of the doctrine of equivalents to the scope of any claims, each numerical parameter should be construed in light of the number of significant digits and ordinary rounding approaches.

The words “comprise”, “comprises”, and “comprising” are to be interpreted inclusively rather than exclusively. Likewise the terms “include”, “including” and “or” should all be construed to be inclusive, unless such a construction is clearly prohibited from the context. The terms “comprising” or “including” are intended to include embodiments encompassed by the terms “consisting essentially of” and “consisting of”. Similarly, the term “consisting essentially of” is intended to include embodiments encompassed by the term “consisting of”. Although having distinct meanings, the terms “comprising”, “having”, “containing” and “consisting of” may be replaced with one another throughout the description of the invention.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

“Typically” or “optionally” means that the subsequently described event or circumstance may or may not occur, and that the description includes instances where said event or circumstance occurs and instances where it does not.

Wherever the phrase “for example,” “such as,” “including” and the like are used herein, the phrase “and without limitation” is understood to follow unless explicitly stated otherwise. In general, the word “instructions,” as used herein, refers to logic embodied in hardware or firmware, or to a collection of software units, possibly having entry and exit points, written in a programming language, such as, but not limited to, Python, R, Rust, Go, SWIFT, Objective C, Java, JavaScript, Lua, C, C++, or C #. A software unit may be compiled and linked into an executable program, installed in a dynamic link library, or may be written in an interpreted programming language such as, but not limited to, Python, R, Ruby, JavaScript, or Perl. It will be appreciated that software units may be callable from other units or from themselves, and/or may be invoked in response to detected events or interrupts. Software units configured for execution on computing devices by their hardware processor(s) may be provided on a computer readable medium, such as a compact disc, digital video disc, flash drive, magnetic disc, or any other tangible medium, or as a digital download (and may be originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. Generally, the instructions described herein refer to logical modules that may be combined with other modules or divided into sub-modules despite their physical organization or storage. As used herein, the term “computer” is used in accordance with the full breadth of the term as understood by persons of ordinary skill in the art and includes, without limitation, desktop computers, laptop computers, tablets, servers, mainframe computers, smartphones, handheld computing devices, and the like.

In this disclosure, references are made to users performing certain steps or carrying out certain actions with their client computing devices/platforms. In general, such users and their computing devices are conceptually interchangeable. Therefore, it is to be understood that where an action is shown or described as being performed by a user, in various implementations and/or circumstances the action may be performed entirely by the user's computing device or by the user, using their computing device to a greater or lesser extent (e.g. a user may type out a response or input an action, or may choose from preselected responses or actions generated by the computing device). Similarly, where an action is shown or described as being carried out by a computing device, the action may be performed autonomously by that computing device or with more or less user input, in various circumstances and implementations.

In this disclosure, various implementations of a computer system architecture are possible, including, for instance, thin client (computing device for display and data entry) with fat server (cloud for app software, processing, and database), fat client (app software, processing, and display) with thin server (database), edge-fog-cloud computing, and other possible architectural implementations known in the art.

Generally, embodiments of the present disclosure are directed towards novel methods, devices, and systems that maintain or reduce internal lens pressure. In particular, the present disclosure relates to usage of light energy (e.g., emitted by one or more lasers) to damage and/or lyse one or more cells and/or proteins in a predetermined area of the lens, more specifically the lens epithelium and adjacent region, such as, for example, a predetermined area in an equatorial region of the lens epithelium.

Such damage and/or lysis non-invasively maintains or reduces internal lens pressure, thereby halting, limiting, reducing, and/or reversing mammalian lens protein aggregation diseases, including, for example, presbyopia and cataracts.

### Lens Aggregation Diseases

Generally, lens aggregation diseases (e.g., presbyopia) manifest clinically in middle age. However, the processes that lead to presbyopia result in a dramatic increase in lens stiffness, which occurs well before the clinical onset of symptoms.

This onset of symptoms is commonly attributed, at least in part, to the loss of natural enzymatic and antioxidant protection in the eye against ultraviolet radiation (e.g., ultraviolet A (UV-A) and ultraviolet B (UV-B) radiation), with a concurrent increase in the production of photochemically active chromophores or oxidants over a period of time.

One skilled in the art will recognize that, as the lens of the eye absorbs light, chromophores are photoactivated and produce reactive oxygen species (e.g., singlet oxygen and superoxide). These oxidants denature lens proteins. At the same time as the lens accumulates such oxidants and/or oxidized components, there is a decreased efficiency of naturally-occurring mechanisms which repair proteins damaged by oxidation.

As a result, oxidative damage can cause progressive hardening of the lens substance, eventually reaching a level where the lens loses its ability to bend in order to focus, the focusing occurring through a process known in the art, and referred to herein, as “accommodation,” and the gradual stiffening causing the loss of ability to bend, which happens approximately between the ages of 42 and continues to around age 57 (known in the art, and referred to herein, as “presbyopia”). Gradual opacification of the lens results in a decreased amount of available light to the retina, thereby resulting in decreasing vision and/or cataract. This occurs, on average, between the ages of 65 and 75. Cataract, if left untreated, will result in surgically reversible blindness.

The cause of various lens aggregation diseases (e.g., presbyopia and cataractogenesis) is believed to be multi-factorial, and influenced by a combination of endogenous oxidation and exogenous oxidation. “Endogenous oxidation” means oxidation that occurs via internal mechanisms such as, for example, oxidation that occurs intraocular photochemical generation of superoxide and its derivatization to other oxidants such as singlet oxygen, hydroxyl radical, hydrogen peroxide and glycation, loss of the lens' natural ultraviolet (UV) filters, and decreasing amounts of natural lens antioxidants, (e.g., glutathione). “Exogenous oxidation” means oxidation due to external and/or environmental factors, such as, for instance, increased exposure to short wavelength and UV radiation, ingestion of chemicals and pollutants, smoking, diseases such as diabetes, and the like.

It is commonly believed in the art that many types of oxidation, including those mentioned previously herein, denature healthy proteins, leading to a gradual loss of lens elasticity, which results in difficulty focusing at close distances (e.g., as occurs in presbyopia) and/or lens opacification (e.g., as results from cataracts).

However, lens aggregation diseases may not be solely, or mainly, initiated by oxidation; thus, such oxidation may not be the root cause of these diseases. While endogenous and exogenous oxidation are factors, other factors that are not recognized and/or under-recognized in the art include the effects on proteins from rising internal lens pressure.

For example, optical clarity of the lens is generally maintained through the process of normal protein folding and unfolding with respect to lens proteins. Healthy proteins fold, unfold, and refold with the help of so-called “molecular chaperones,” a term known in the art for proteins that assist in healthy folding and/or unfolding. Proteins may misfold and/or unfold due to a variety of factors, such as, for instance, oxidation, declining amounts of chaperones, and other factors. Misfolding may lead to protein aggregation, which, with respect to lens aggregation diseases, gradually hardens and opacifies the lens, thereby causing and/or exacerbating such diseases (e.g., presbyopia, cataracts).

Thus, conventional solutions have failed to adequately consider factors and/or changes other than exogenous and endogenous oxidation that may contribute to the onset and/or development of lens aggregation diseases.

### Lens Pressure

Purely as a non-limiting example, changes in internal lens pressure may contribute to lens protein misfolding and/or unfolding, thereby leading to a loss of lens flexibility and the concomitant reduction in ability of the lens to focus.

It should be appreciated that, once any cellular protein is folded, various stress conditions may pose a threat to the protein's integrity. For instance, temperature variations, pressure, osmotic changes, antibiotics, solvents, and other chemicals and/or forces not only interfere with transcription, translation, and protein folding, but can also often disrupt the accurate three-dimensional protein structure.

It is generally known that pressure affects proteins, and more specifically, that pressure can unfold proteins. See, e.g., P. W. Bridgman, “The coagulation of albumin by pressure,” J Biol. Chem. 19:511-12 (1914); W. Kauzmann, “Thermodynamics of unfolding,” Nature 325:763-64 (1987).

Specifically, with respect to lens aggregation diseases, pressure may lead to precipitation and aggregation of proteins (e.g., lens proteins), cross-linking via disulfide bonds, reduction of glutathione, phosphorylation, and other post-translational protein modification factors that may cause progression of presbyopia and cataract.

Various sources may cause such pressure inside the lens, leading to elevated internal lens pressure, as described in further detail below.

### Hydrostatic Pressure

The space between the fiber cells of the lens is known as the interstitium. Fluid within the interstitium is termed the interstitial fluid. Such fluid circulates through the lens and fiber cells of the lens are accordingly bathed by, and within, the interstitial fluid. An intracellular gradient of hydrostatic pressure drives fluid from central fiber cells outward towards surface epithelial cells, pushing outwards against the lens capsule.

Mathematically, hydrostatic pressure is defined as a change in volume divided by a change in pressure. As a result, the more fluid that filters into the interstititum, the greater the volume of the interstitial space (Vi) and the hydrostatic pressure within that space (Pt). For instance, in young, healthy human lenses, an intracellular hydrostatic pressure gradient is from ˜340 mmHg in central fiber cells to ˜0 mmHg in surface cells.

Intralenticular (that is, located within the lens of the eye) hydrostatic pressure generally increases with age. Generally, as a consequence of accommodation, there is a tendency for water to move from the lens to the surrounding area(s), which then increase the osmolarity of the lens. The ratio of free water to bound water decreases with increasing pressure, and accordingly increases with decreasing pressure.

Accommodation often declines between the ages of forty and sixty. In the oldest normal human lenses, an increase in osmotic pressure causes the release of bound water to become free water. When such a response to pressure is irreversible, the released free water accumulates in so-called lakes.

This release of bound water from the hydration layers of macromolecules and its conversion to free water in condensed systems is known as syneresis, which is known as the extraction or expulsion of a liquid from a gel. In the lens, decreasing osmotic pressure induces syneresis.

During accommodation, liquid is expelled from its bound state with lens crystallins, thereby becoming free water, thus decreasing osmotic pressure. As the ability to accommodate is lost, the free water-to-bound water ratio decreases with increasing pressure, resulting in a significant syneretic response. The ability of the human lens to respond reversibly to pressure decreases with a decrease in accommodation. When the accommodation ability is lost altogether, an increase in free water, which may be a source of cataract formation, may ensue.

Younger lenses convert free water to bound water efficiently with increasing hydrostatic pressure, but, in older lenses, this ability is diminished and, in some cases, reversed. Generally, the total water content is much higher in cataractous lenses (that is, lenses affected by cataract) than normal lenses. Thus, in complete presbyopia (i.e., where there is no accommodation), the lens is fixed in its unaccommodated, compressed configuration, with a lower tendency for water movement out of the interstitium, thereby creating higher internal pressure. Therefore, with aging, the ability of the lens to compensate for increased hydrostatic pressure is decreased.

### Compression

Another factor that may result in elevated internal lens pressure is compression that results via physical constraint of the lens.

Hydrostatic pressure affects other organs in the body besides the eye, including, for instance, the brain (which is encased by rigid bone) and the kidney (which is encased by a capsule). The lens, like the kidney, is confined by a capsule, and exists in a state in which small increases in fluid volume leads to large increases in pressure. Large increases in the interstitial pressure of tissue can lead to tissue damage and cellular death. Accordingly, constraint of the capsule surrounding the lens may add to increasing internal lens pressure.

In primates, the lens capsule itself is generally a strong, transparent membrane that is capable of shaping the lens and its surface curvature by participating in the process of accommodation. The capsule is an uninterrupted basement membrane completely enclosing the lens, sequestering the lens from other ocular tissues, protecting its optical integrity from penetration by large molecules and protecting the lens from infectious microbes (e.g., viruses, bacteria).

As the lens is avascular, the capsule must also allow for the passive exchange of metabolic substrates and waste in and out of the lens.

The elastic modulus of the capsule must therefore be sufficiently higher than that of the lens substance in order to allow the forces applied by the ciliary muscles to mold the lens shape. The adult human lens capsule has an elastic modulus of approximately two thousand times higher than the cellular lens cortex and nucleus that it surrounds.

During accommodation, the zonules, which insert into the lens capsule, apply stress that has both parallel (e.g., stretching) and perpendicular (e.g., compressive) components. These discrete stresses are transformed by the capsule into a uniform stress that is approximately perpendicular to the lens surface. The transition from the unaccommodated to the accommodated state would include a reduction of stresses perpendicular to the lens surface.

Under uniaxial load, capsular elastic moduli at 10% strain increase with age until about age thirty-five, from around 0.3 N/mm² to 2.3 N/mm², and then becomes relatively constant thereafter. In other words, past age thirty-five, the capsule load is maximized at around 2.3 N/mm².

On top of this, continual production of lens cell fibers by the lens epithelium in the environment constrained by the lens capsule, which is fixed in volume, accordingly contributes to continual crowding and compaction. The fluidic changes combined with continued pressure from the production of proteins in a confined space, result in increasing hydrostatic pressure with age and a syneretic process that continually increases resistance within the lens. This, in turn, leads to increased light scattering and a less pliable lens, decreasing the ability of the lens to accommodate, as seen in, e.g., presbyopia and other diseases.

Over time, there is an increase in lens stiffness and elastic modulus observed in the lens nucleus and cortex, resulting from the continual accession of fiber cells. As the elastic modulus of the lens substance increases, more force must be transmitted through the lens capsule to mold its shape. The inability of the lens capsule to achieve a sufficient elastic modulus over the lens substance in order to transmit the necessary forces for accommodation may be a key cause of presbyopia.

### Maintaining Lens Flexibility

Accordingly, embodiments of the present disclosure are directed to novel methods, devices, and systems that maintain or reduce internal lens pressure, by, e.g., maintaining lens flexibility. Without wishing to be bound by theory, a reduction in internal lens pressure may protect the lens from forming post-translational modifications, thereby treating (e.g., retarding, eliminating or reversing) presbyopia and/or cataract.

Generally, human tissue absorbs photon energy. Such energy, as radiant energy, can be reemitted or transformed into heat, and thereby increase the internal temperature of the tissue. If the tissue is warmed past a certain temperature, the heat energy can disrupt cellular function, and even damage or destroy the tissue.

The application of light energy for photomanipulation of human tissue is generally known in the field of ophthalmology. Laser light may be used as the source of light energy, although other light energies may also be used. Laser light is understood as light which is sufficiently monochromatic to allow sufficient focus. One non-limiting example of the application of laser light is shown in U.S. Pat. No. 6,322,556, where laser light is applied to ablate and thereby remove small portions of the lens with the purpose of correcting vision. Further, U.S. Pat. No. 6,726,679 describes the application of laser light to dissolve opacities and/or hardenings of an unopened eye.

In at least one example of the present disclosure, light energy is utilized to disrupt one or more portions of the lens epithelium, such as, for instance, in the periphery, and more specifically, in the germinative zone. The light energy, which may be provided by, for example, one or more lasers, disrupt the epithelium's ability to continually produce proteins, thereby lessening compaction and/or internal lens pressure.

In at least a further example, the light energy is focused specifically on the lens epithelial cells, leaving the overlying capsule and zonules intact.

In at least one example, specific doses of light (e.g., which may be provided as one or more pulses of light) are applied to one or more specific positions within the eyes (e.g., by an automated therapeutic instrument), thereby avoiding ineffective under-treatment or damaging over-treatment (e.g., gas blisters, also known as cavitation bubbles), which might otherwise result with set, non-adjustable values of the light energy and/or the laser. For example, the aforementioned blisters may occur due to, e.g., local evaporation of constituent molecules and/or fluid in the lens. See, e.g., U.S. Pat. No. 6,726,679. The appearance and collapse of these blisters may induce significant mechanical stress on the lens and/or surrounding tissue.

It should be appreciated that the aforementioned specific doses of light, as well as the exact positioning of the light within the lens, may vary from patient to patient due to natural differences in eye structure and composition.

It should further be appreciated that one or more treatment areas for the light energy and/or the laser are located close to the zonules of Zinn (also known in the art as Zinn's membrane or the ciliary nodule), which are elastic fibrils that hold the lens in place. Light energy that is focused or disposed too close to the zonules of Zinn may damage or break them, leading to loss of lens elasticity, and possibly even subluxation of the lens. However, it should be appreciated that, in at least one example, unfocused light energy can be directed through the zonules without damaging them. In such an example, the unfocused light energy may be focused at one or more positions after passing through the zonules.

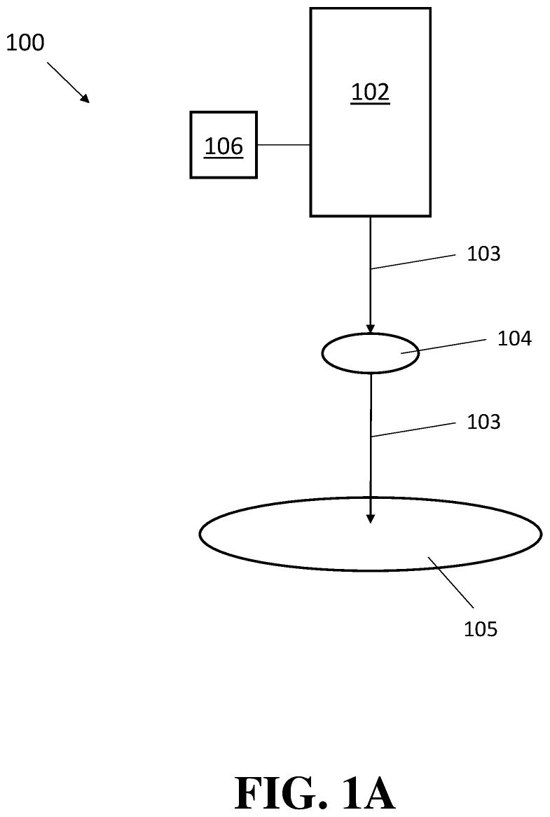

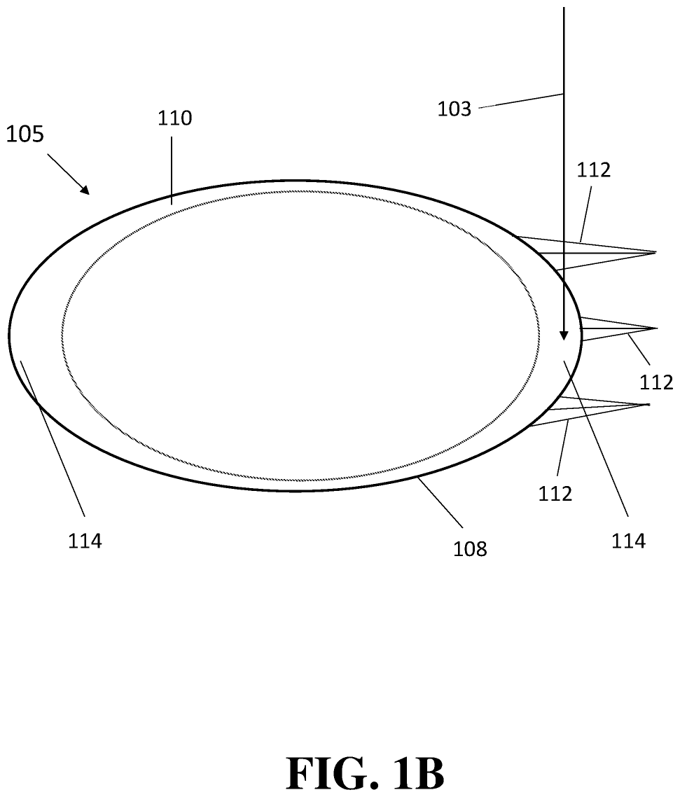

In at least one example, a system 100 for non-invasive or minimally invasive photomanipulation of the lens epithelium of an animal or human eye is disclosed, as shown in FIG. 1A. The system comprises a source of light energy 102 (e.g., a laser) emitting one or more beams 103 of light energy, a focuser 104 for focusing the one or more beams of light energy into a predetermined area of the eye 105 (e.g., a predetermined area of the lens epithelium), an adjuster 106 for adjusting at least one parameter (e.g., focus, intensity, wavelength, pulse length, repetition frequency, and/or pulse train length) of the one or beams of light energy. Thus, the one or more beams of light energy manipulate and/or disrupt tissue in the predetermined area of the eye, as shown in further detail in FIG. 1B.

FIG. 1B is a diagram showing portions of the eye 105, including the lens capsule 108, the lens epithelium 110, and the zonules 112. The lens epithelium 110 includes the germinal zones 114. In at least one example, the one or more beams of light energy 103 are directed at a predetermined area of the lens epithelium 110, and, in particular, one or more portions of the germinal zones 114. As mentioned above herein, focusing the one or more beams of light energy on the germinal zones avoids potentially detrimental impacts of the light energy on either the surrounding lens capsule 108 or the zonules 112.

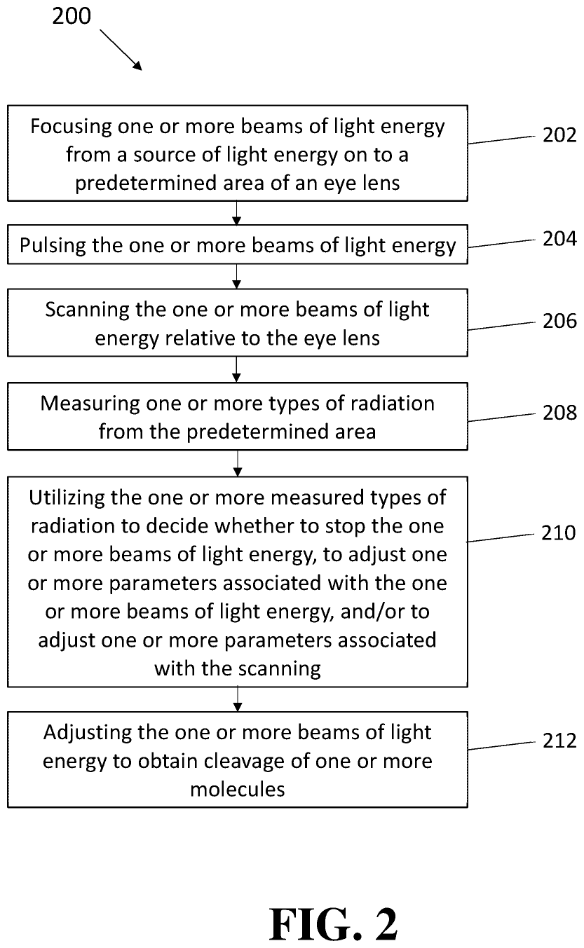

In at least a further example a method 200 is disclosed for non-invasive or minimally invasive photomanipulation of the lens epithelium of an animal or human eye, as shown in FIG. 2. The method 200 may include focusing one or more beams of light energy from a source of light energy (e.g., a laser) on to a predetermined area of an eye lens at block 202, pulsing the one or more beams of light energy at block 204, scanning the one or more beams of light energy relative to the eye lens at block 206, measuring one or more types of radiation from the predetermined area at block 208, utilizing the one or more measured types of radiation to decide whether to stop the one or more beams of light energy, to adjust one or more parameters associated with the one or more beams of light energy, and/or to adjust one or more parameters associated with the scanning at block 210.

The one or more parameters associated with the one or more beams of light energy may include, for instance, focus, intensity, wavelength, pulse length, repetition frequency, and/or pulse train length. The one or more parameters associated with the scanning may include, for example, scan velocity, size of scanned volume, scan repetitions, and scan pattern.

The method 200 may additionally include adjusting the one or more beams of light energy to obtain cleavage of one or more molecules at block 212. These one or more molecules may be, for instance, lens proteins, lens protein cross-links, macromolecular adducts, and the like. The aforementioned cleavage occurs without damage to healthy lens proteins, cell membranes, and/or other components of the lens other than the aforementioned predetermined area. Thus, the method avoids or minimizes cavitation, mechanical effects, acoustic effects, and/or thermal effects on cells, molecules, and/or components that are not a treatment target and/or are outside the predetermined area.

Additional embodiments of the methods, devices, and/or systems mentioned above herein may include one or more of the following, in any mutually non-exclusive combination.

The one or more beams of light energy may be provided as one or more pulses. Additionally, the light energy may be focused on one or more points within the equatorial region of the lens epithelium. This equatorial region may include one or more portions of the lens epithelium and/or boundary regions between the lens epithelium and the lens fiber core. The one or more points may be arranged in one or more patterns (e.g., in a line or in an arc).

In at least a further example, the one or more beams of light energy damage, lyse or kill one or more cells in the lens epithelium. Alternatively or additionally, in at least one embodiment, the one or more beams of light energy damage one or more cells in the lens epithelium without lysing, damaging or killing the one or more cells.

In at least an additional example, the light energy (e.g., from the one or more beams of light energy) is delivered either above or below the predetermined area of the lens epithelium such that heat dissipates into the predetermined area, thereby resulting in damage and/or lysis of one or more cells in the predetermined area.

The light energy may be provided by, for example, one or more neodymium-doped yttrium aluminum garnet (Nd:YAG) lasers. The light energy provided by the one or more Nd:YAG lasers can be focused (e.g., by the aforementioned focuser) into a portion of the lens epithelium under the lens capsule. A skilled artisan will appreciate that focusing of such light energy on to, or into, the capsule itself could result in damaging the zonules.

In at least one example, the one or more beams of light energy provide, in total, less than one hundred and seventy-five millinewtons (mN) to the predetermined area of the lens epithelium. In at least an additional embodiment, the one or more beams of light energy provide, in total, less than one hundred and ten mN to the predetermined area.

In further examples, the light energy may be provided by one or more of: an excimer laser, a femtosecond laser, a femtosecond multi-shooting (FSMS) laser, a holmium yttrium aluminum garnet (YAG) laser, a potassium-titanyl-phosphate (KTP):YAG laser, a ytterbium (Yb):YAG laser, a metal vapor laser, a carbon dioxide (CO₂) laser, a ruby laser (e.g., at a wavelength of 694 nanometers (nm)), an argon (Ar) laser (e.g., at wavelengths of 488 and/or 514 nm), a helium (He)-neon (Ne) laser (e.g., at a wavelength of 632.8 nm), a krypton laser (e.g., at wavelengths of 521, 530, 568, and/or 647 nm), a gallium (Ga)-aluminum (Al)-arsenide (As) laser (e.g., at wavelengths of 650 and/or 805 nm), a Ga—As laser (e.g., at a wavelength of 904 nm), an erbium (Er):glass laser, a diode laser, a pumped-dye laser, a pulsed gas laser, a thermal laser, a thermal and mechanical laser, a plasma, thermal, and mechanical laser, a thermal and photochemical laser, and a photoablative laser.

Further, the light energy may be in a wavelength range of between 1000 and 1500 nm, or between 450 and 550 nm.

The one or more lasers providing the light energy may have an output power of, for example, greater than 500 milliwatts (mW) or 0.5 Watts (e.g., corresponding to Class IV lasers).

The output of the one or more lasers may be monochromatic. Alternatively or additionally, the output may be coherent and parallel.

In a non-limiting example, the light energy is provided by a photodynamic (photoradiation) or photocoagulation source (e.g., dye laser), and the light energy is transmitted through refractive media in the lens, thereby resulting in selective destruction of eye tissue (e.g., lens epithelial tissue). It should therefore be appreciated that one or more lasers that utilize a photochemical mechanism, as opposed to an ablative or thermal mechanism, can be used. This results in the target tissue (e.g., lens epithelial tissue) absorbing the light energy, causing a chemical change.

In further example, the light energy may be provided as photodynamic therapy (PDT) and/or photothermal therapy (PTT). A skilled artisan will recognize that PDT generally uses one or more photosensitizer molecules and/or drugs in conjunction with light energy, while PTT generally utilizes one or more photothermal agents in conjunction with selective local heating of tissues. Accordingly, in at least one example, PDT (e.g., light energy as mentioned herein in conjunction with bacteriochlorin a (BCA)) is used, in whole or in part, to damage and/or lyse lens epithelial cells. In at least an additional example PTT (e.g., light energy as mentioned herein in conjunction with one or more localized light-absorbing dyes) is used, in whole or in part, to damage and/or lyse lens epithelial cells.

One or more examples may also comprise instructions executed by at least one processor to operate one or more of the sources of light energy described above herein. As a non-limiting example, such instructions may be used to move, focus, scan, and/or adjust one or more lasers to manipulate a predetermined area of the lens epithelium. As a further non-limiting example, the instructions may be used to operate one or more steps of the method 200.

Accordingly, embodiments of the present disclosure provide novel methods, devices, and systems for non-invasive photomanipulation (e.g., via laser-mediated light energy) to one or more portions of an eye lens (e.g., the lens epithelium in the equatorial region). Such embodiments provide non-disruptive or minimally-disruptive amounts of photonic energy to other areas of the lens and/or eye, thereby ensuring effective treatment of lens aggregation diseases (e.g., presbyopia).

These and other objectives and features of the invention are apparent in the disclosure, which includes the above and ongoing written specification.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated.

The invention is not limited to the particular embodiments illustrated in the drawings and described above in detail. Those skilled in the art will recognize that other arrangements could be devised. The invention encompasses every possible combination of the various features of each embodiment disclosed. One or more of the elements described herein with respect to various embodiments can be implemented in a more separated or integrated manner than explicitly described, or even removed or rendered as inoperable in certain cases, as is useful in accordance with a particular application. While the invention has been described with reference to specific illustrative embodiments, modifications and variations of the invention may be constructed without departing from the spirit and scope of the invention as set forth in the following claims.

I/We claim:

1. A method for minimally invasive disruption of lens epithelium of an animal or a human eye, the method comprising:
focusing one or more beams of light energy from one or more lasers on to a germinative zone located in a periphery of the lens epithelium; and
pulsing the one or more beams, thereby inducing photochemical reactions to cause damage and/or lysis of one or more cells in the germinative zone,
wherein the one or more lasers each have a power output of greater than 0.5 Watts, and
wherein the one or more focused beams provide a total of less than 110 millinewtons (mN) of force.

2. The method of claim 1, further comprising focusing the one or more beams of light energy from the one or more lasers on to one or more points in an equatorial region of the lens epithelium.

3. The method of claim 1, wherein the light energy is delivered above and/or below a predetermined area such that heat from the light energy dissipates into the predetermined area, thereby resulting in damage to, and/or lysis of, one or more cells in the predetermined area.

4. The method of claim 1, further comprising adjusting at least one parameter of the one or more beams, wherein the at least one parameter is selected from the group consisting of: beam focus, beam intensity, wavelength of the light energy, pulse length of the pulses, repetition frequency of the pulses, pulse train length of the pulses, and combinations thereof.

5. The method of claim 1, wherein the one or more lasers are selected from the group consisting of: a neodymium-doped yttrium aluminum garnet (Nd:YAG) laser, an excimer laser, a femtosecond laser, a femtosecond multi-shooting (FSMS) laser, a holmium yttrium aluminum garnet (YAG) laser, a potassium-titanyl-phosphate (KTP):YAG laser, a ytterbium (Yb):YAG laser, a metal vapor laser, a carbon dioxide (CO₂) laser, a ruby laser, an argon (Ar) laser, a helium (He)-neon (Ne) laser, a krypton laser, a gallium (Ga)-aluminum (Al)-arsenide (As) laser, a Ga—As laser, an erbium (Er):glass laser, a diode laser, a pumped-dye laser, a pulsed gas laser, a thermal laser, a thermal and mechanical laser, a plasma, thermal, and mechanical laser, a thermal and photochemical laser, a photoablative laser, and combinations thereof.

6. The method of claim 1, wherein the light energy has a wavelength of between 1000 and 1500 nm or between 450 and 550 nm.

7. The method of claim 1, further comprising using, in conjunction with the one or more beams, one or more photosensitizer molecules and/or one or more photothermal agents.

8. A method for manipulation of lens epithelium of an animal or a human eye, the method comprising:
focusing one or more beams of light energy from one or more lasers on to a germinative zone located in a periphery of the lens epithelium;
pulsing the one or more beams; and
scanning the one or more beams relative to the lens epithelium,
wherein the one or more focused beams provide a total of less than 175 millinewtons (mN) of force.

9. The method of claim 8, further comprising:
measuring one or more types of radiation from the germinative zone; and
utilizing the one or more measured types of radiation to decide whether to stop the one or more beams, to adjust one or more parameters associated with the one or more beams, and/or to adjust one or more parameters associated with the scanning.

10. The method of claim 9, wherein the one or more parameters associated with the one or more beams is selected from the group consisting of: beam focus, beam intensity, wavelength of the light energy, pulse length, repetition frequency, pulse train length, and combinations thereof, and
wherein the one or more parameters associated with the scanning is selected from the group consisting of: scan velocity, size of scanned volume, scan repetitions, scan pattern, and combinations thereof.

11. The method of claim 8, wherein the one or more beams provide less than 110 millinewtons (mN) of force to the germinative zone.

12. The method of claim 8, wherein the one or more lasers are selected from the group consisting of: a neodymium-doped yttrium aluminum garnet (Nd:YAG) laser, an excimer laser, a femtosecond laser, a femtosecond multi-shooting (FSMS) laser, a holmium yttrium aluminum garnet (YAG) laser, a potassium-titanyl-phosphate (KTP):YAG laser, a ytterbium (Yb):YAG laser, a metal vapor laser, a carbon dioxide (CO₂) laser, a ruby laser, an argon (Ar) laser, a helium (He)-neon (Ne) laser, a krypton laser, a gallium (Ga)-aluminum (Al)-arsenide (As) laser, a Ga—As laser, an erbium (Er):glass laser, a diode laser, a pumped-dye laser, a pulsed gas laser, a thermal laser, a thermal and mechanical laser, a plasma, thermal, and mechanical laser, a thermal and photochemical laser, a photoablative laser, and combinations thereof.

13. The method of claim 12, wherein the light energy has a wavelength of between 1000 and 1500 nm or between 450 and 550 nm, and wherein the one or more lasers each have a power output of greater than 0.5 Watts.

14. A system for laser-mediated disruption of lens epithelium of an animal or a human eye, the system comprising:
one or more lasers emitting one or more beams of light energy;
a focuser for focusing the one or more beams on to a predetermined area in the lens epithelium, thereby causing damage and/or lysis of one or more cells in the predetermined area; and
an adjuster for adjusting at least one parameter of the one or more beams,
wherein the one or more lasers each have a power output of greater than 0.5 Watts, and
wherein the one or more focused beams provide a total of less than 175 millinewtons (mN) of force.

15. The system of claim 14, wherein the light energy is provided in one or more pulses, and wherein the at least one parameter is selected from the group consisting of: beam focus, beam intensity, wavelength of the light energy, pulse length of the one or more pulses, repetition frequency of the one or more pulses, pulse train length of the one or more pulses, and combinations thereof.

16. The system of claim 14, wherein the predetermined area is located in a germinative zone in a periphery of the lens epithelium.

17. The system of claim 14, wherein the light energy is focused on one or more points in an equatorial region of the lens epithelium.

18. The system of claim 14, where the one or more lasers comprises a neodymium-doped yttrium aluminum garnet (Nd:YAG) laser, and wherein the predetermined area is located in a portion of the lens epithelium underneath the lens capsule.

19. The system of claim 14, wherein the one or more focused beams cleave one or more molecules in the predetermined area.

20. The system of claim 14, wherein the one or more focused beams provide a total of less than 110 millinewtons (mN) of force.
